(19) United States
(12) Patent Application Publication
(10) Pub. No.: US 2025/0312262 A1
(43) Pub. Date: Oct. 9, 2025

(54) TATTOO INK

(71) Applicant: VEIL VariEty In coLours GmbH, Hamburg (DE)

(72) Inventor: Maximilian Tim Wolde, Hamburg (DE)

(21) Appl. No.: 18/628,176

(22) Filed: Apr. 5, 2024

(51) Int. Cl. A61K 8/73; A61K 8/92; A61Q 1/00; A61Q 1/02

(52) U.S. Cl. CPC A61K 8/735; A61K 8/922; A61Q 1/00; A61Q 1/025; A61K 2800/91

(57) ABSTRACT

The invention relates to a tattoo ink with improved skin compatibility based on a dispersion of at least one pigment in an aqueous carrier liquid containing a ginger oil and/or cajeput oil and hyaluron and/or one or more hyaluron derivatives, its use and a process for the production of tattoos.

### BACKGROUND

The invention relates to a tattoo ink (often also referred to as tattoo color or tattooing agent) with improved skin compatibility.

A tattoo is a motif that is applied to the skin of a living being using tattoo ink or other coloring agents. The motif is arbitrary and can be a monochrome or multicolored image, sign or symbol, pattern, ornament or even text. Tattoos can be applied to humans and animals.

Human tattoos can be used as body ornaments, permanent make-up, jewelry, signs of belonging to a group, as a statement or code, or as a fashion accessory. Tattoos can also be used in the medical field, for example to cover scar tissue or for breast reconstruction. For pets and breeding animals, tattoos are usually used for labelling and/or identification.

Depending on the desired effect, a tattoo ink is applied to the second layer of skin (the dermis) using one or more needles. While the top layer of skin above is constantly renewed, the dermis is not. The color pigments from the tattoo ink are permanently stored there so that the tattooed motif remains permanently visible. Tattooing is a puncturing of the skin, whereby a coloring agent is introduced into the skin at the same time as the puncturing using the tattoo ink.

Tattoo studios usually use an electric tattoo machine for tattooing. Specially sharpened needle tips are used to pierce the top layer or epidermis of the skin and the tattoo ink is applied to the second layer or dermis. The puncture must not be superficial or too deep. If the tattoo ink is only applied to the epidermis, the coloring agent would be gradually removed due to the constant renewal of this skin layer. If the tattoo is too deep, the coloring agent will be washed out due to bleeding. Therefore, when tattooing, it is necessary to adjust the penetration depth depending on the skin type and the area of the body to be tattooed.

Conventional tattoo inks comprise one or more coloring agents, one or more carrier liquids and, if necessary, auxiliary substances. Carrier liquids are solvents and/or dispersants such as distilled water or alcohols.

Tattoo inks contain color pigments, such as metal salts, as coloring agents. These can be water-soluble, but are often hydrophobic and therefore not water-soluble, meaning that solubilisers, solubilizing agents and/or dispersing agents are used to disperse the pigments in water.

As the tattoo ink remains in the body to a large extent, its quality is of particular importance.

One problem with tattooing is that the penetration of the needle into the skin is perceived as unpleasant or even painful and also often leads to skin reactions. There has therefore been no shortage of attempts to develop tattoo inks that reduce pain and skin reactions during and after the tattooing process.

For example, WO 2019/057822 A1 describes a combination for tattooing, comprising a tattoo ink and one or more local anesthetics for the skin.

KR102084261B1 discloses a tattoo ink and a process for the preparation thereof, wherein the tattoo ink contains various plant extracts which are intended to impart antibacterial, anti-inflammatory, and pain-relieving properties to the tattoo ink.

Nevertheless, there is still a need for tattoo inks that further reduce pain and skin reactions during and after the tattooing process.

### SUMMARY

The task of the invention is therefore to provide tattoo inks that are harmless to health and further reduce pain and skin reactions during and after the tattooing process.

In one aspect, a tattoo ink based on a dispersion of at least one pigment in an aqueous carrier liquid is disclosed. The tattoo ink contains a combination of at least one oil selected from a ginger oil and cajeput oil and hyaluron and/or one or more hyaluron derivatives.

In another aspect, a method for the production of tattoos is disclosed. The method includes a step on applying a suitable amount of a tattooing ink containing a combination of at least one oil selected from a ginger oil and cajeput oil and hyaluron and/or one or more hyaluron derivatives to the needles of a tattooing machine. The tattooing machine may be a magnetic coil machine or a rotary tattooing machine. The tattooing ink is introduced into the skin of a living being by means of the tattooing machine.

### DETAILED DESCRIPTION

The tattoo ink according to the invention is based on a dispersion of at least one pigment in an aqueous carrier liquid and contains a combination of at least one oil, which is selected from a ginger oil and cajeput oil, and hyaluron and/or one or more hyaluron derivatives. The sum of the proportions of the at least one oil and of the hyaluron and/or one or more hyaluron derivatives in the tattoo ink according to the invention is preferably 0.1 to 11% by weight, in particular 1 to 8% by weight and particularly preferably 2.5 to 7% by weight. Specifically, the tattoo ink contains 0.05 to 10% by weight, preferably 0.05 to 7% by weight and in particular 0.5 to 5% by weight ginger oil and/or 0.01 to 10% by weight, preferably 0.01 to 7% by weight and in particular 0.5 to 3% by weight ginger oil and 0.01 to 10% by weight, preferably 0.01 to 7% by weight and in particular 0.5 to 5% by weight hyaluron and/or one or more hyaluron derivatives.

In a particularly preferred embodiment, the tattoo ink according to the invention contains a ginger oil, cajeput oil and hyaluron and/or one or more hyaluron derivatives.

Ginger oil refers to essential oils that have been extracted from ginger root. Such oils are commercially available in cosmetic quality from numerous suppliers. According to the invention, the oil of ginger cassumunar, also known as plai oil, is particularly preferred.

Cajeput oil is an essential oil extracted from the leaves and twig tips of the cajeput tree (Malealeuca leucadendra). This essential oil is also commercially available in cosmetic quality.

Hyaluron refers to hyaluronic acid and its physiologically compatible salts. Preferably, sodium hyaluronate is used according to the invention.

All molecular weights that can be produced biotechnologically can be used, preferably hyaluronic acid with a molecular weight of <5 kilodaltons, 10-50 kilodaltons, 100-300 kilodaltons, 400-800 kilodaltons, 1200-1800 kilodaltons and over 2200 kilodaltons.

Hyaluronic acid derivatives means compounds of hyaluronic acid and in particular hydroxypropyl trimonium hyaluronate, hydrolysed sodium hyaluronate, vegan hyaluronic acid Tremella Fuciformis Sporocarp Extract, sodium acetylated hyaluronate and sodium hyaluronate crosspolymer When using hyaluronate derivatives, it can be advantageous according to the invention if the tattoo ink additionally contains N-acetylglucosamine, a building block of hyaluronic acid. In this embodiment, the proportion of N-acetylglucosamine in the tattoo ink is preferably 0.5 to 5% by weight.

In addition, the tattoo ink according to the invention may contain conventional auxiliary substances in a proportion of up to 30% by weight. These are preferably humectants, in particular pentylene glycol or panthenol, dispersing agents, in particular polyhydroxystearic acid, binding agents, thickening agents, viscosity regulators, preservatives, stabilizers, emulsifiers, antioxidants or agents for regulating the pH value.

The aqueous carrier liquid is water, a vegetable hydrolate or a mixture of water or a vegetable hydrolate and one or more physiologically harmless solvents, in particular lower saturated alcohols or particularly preferably glycerine, the proportion of water or vegetable hydrolate in the mixture preferably being at least 70% by weight and in particular at least 80% by weight. The proportion of the aqueous carrier liquid in the tattoo ink is preferably 10 to 98% by weight, particularly preferably 30 to 90% by weight and especially 40 to 70% by weight. It is expedient to use the aqueous carrier liquid and its components in sterile form.

The proportion of the at least one pigment in the tattoo ink is preferably 1 to 90% by weight, particularly preferably 5 to 40% by weight and especially 10 to 40% by weight.

Suitable pigments according to the invention are inorganic color pigments and white pigments from the group of titanium dioxide, zinc oxides and boron nitride. Preferred pigments are iron oxides, aluminum oxide, zirconium oxides, cobalt oxides, cerium oxides, nickel oxide, nickel-chromium oxides, as well as composite oxides or calcium hydroxide, iron hydroxide, aluminum hydroxide, chromium hydroxide, magnesium hydroxide and composite metal hydroxides, as well as ultramarine blue, iron sulfides, manganese violet, carbon black, mica or kaolin.

In one embodiment of the invention, a white pigment from the group consisting of titanium dioxide, zinc oxides and boron nitride is used as a carrier pigment in combination with at least one inorganic color pigment. Boron nitride is particularly preferred as a white pigment.

Carrier pigment means that the carrier pigment is coated with the inorganic color pigment.

A tattoo ink according to the invention can preferably have the following composition: 40 to 70% by weight of aqueous carrier liquid, 2.5 to 8% by weight of the combination of ginger oil, cajeput oil and hyaluronic acid 10 to 40% by weight of at least one pigment 0 to 15% by weight of excipients.

The composition of the tattoo ink according to the invention can be adapted to different tattooing requirements over a wide range. In particular, the viscosity can be adjusted over a wide range.

The combination of ginger oil and/or cajeput oil and hyaluronic acid and/or one or more hyaluronic acid derivatives improves skin regeneration after the tattooing process and has a decongestant and anti-inflammatory effect. Particularly favorable effects are achieved if the tattoo ink contains a ginger oil, in particular plai oil and/or cajeput oil, and hyaluron and/or one or more hyaluron derivatives.

The tattoo ink according to the invention is particularly suitable for producing a permanent tattoo on the skin of a living being, in particular a human being.

In addition, the tattoo ink according to the invention is also suitable for use as a “leave-on” ink for temporarily coloring the skin of a living being, in particular a human being, or parts thereof.

The tattoo ink according to the invention can be produced in a manner known to the skilled person by mixing the components.

Another object of the invention is a method for producing tattoos, in which a suitable amount of the tattoo ink according to the invention is applied to the needles of a tattoo machine, in particular a magnetic coil machine or a rotary tattoo machine, and is introduced into the skin of a living being, in particular a human being, by means of the tattoo machine.

Further advantages, special features and useful further embodiments of the invention are shown in the following examples.

| | Example 1 | Example 2 | Example 3 | Example 4 | Example 5 |
|---|---|---|---|---|---|
| Raw material | Weight % | Weight % | Weight % | Weight % | Weight % |
| Glycerine 99.5% Vegan | 6 | 5 | 5 | 5 | 5 |
| Water, demin. | 41 | 45.5 | 45.5 | 45.5 | 32.5 |
| Plai oil | 1 | 1 | 1 | 1 | 4 |
| Cajeput oil | 1 | 1 | 1 | 2 | 1 |
| Pentylene glycol | 5 | 8 | 8 | 5 | 8 |
| Panthenol | 5 | 3 | 3 | 3 | 3 |
| Sodium hyaluronate <50 | 5 | 0.5 | 0.5 | 2.5 | 0.5 |
| kilodaltons | | | | | |
| Polyhydroxystearic acid | 1 | 1 | 1 | 1 | 1 |
| Iron oxide black | 35 | | | | |
| CI 77499 | | | | | |
| Cromoxide green CI | | 35 | | | |
| 77288 | | | | | |
| Zinc oxide CI 77947, | | | 35 | | |
| boron nitride | | | | | |
| Iron oxide yellow CI | | | | 35 | |
| 77492 | | | | | |
| Ultramarine blue | | | | | 45 |
| CI 77007, | | | | | |
| kaolin | | | | | |

| | Example 6 | Example 7 | Example 8 | Example 9 | Example 10 |
|---|---|---|---|---|---|
| Raw material | Weight % | Weight % | Weight % | Weight % | Weight % |
| Glycerine 99.5% Vegan | 6 | 5 | 5 | 5 | 5 |
| Water, demin. | 45 | 45.5 | 45 | 45.5 | 22.75 |
| Plai oil | 1 | 1 | 1 | 1 | 4 |
| Cajeput oil | 1 | 1 | 1 | 2 | 1 |
| Pentylene glycol | 5 | 8 | 8 | 5 | 8 |
| Panthenol | 5 | 3 | 3 | 3 | 3 |
| Sodium hyaluronate <50 | | 0.5 | | | |
| kilodaltons | | | | | |
| Sodium hyaluronate | 1 | | | | |
| 1800 kilodaltons | | | | | |
| Sodium hyaluronate | | | 1 | | |
| 400 kilodaltons | | | | | |
| Hydrolysed sodium | | | | 2.5 | |
| hyaluronate | | | | | |
| Sodium hyaluronate | | | | | 0.25 |
| crosspolymer | | | | | |
| Polyhydroxystearic acid | 1 | 1 | 1 | 1 | 1 |
| Iron oxide black | 35 | | | | |
| CI 77499 | | | | | |
| Iron oxide red CI 77491, | | 35 | | | |
| Iron oxide black | | | | | |
| CI 77499 | | | | | |
| Zinc oxide CI 77947, | | | 35 | | |
| boron nitride | | | | | |
| Iron oxide yellow CI | | | | 35 | |
| 77492 | | | | | |
| Ultramarine blue | | | | | 55 |
| CI 77007, | | | | | |
| kaolin | | | | | |
| | Example 11 | Example 12 | Example 13 | Example 14 | Example 15 |
| Raw material | Weight % | Weight % | Weight % | Weight % | Weight % |
| Glycerine 99.5% Vegan | 6 | 5 | 5 | 5 | 5 |
| Water, demin. | 45 | 53.5 | 33 | 40.5 | 42.75 |
| Plai oil | 0.5 | 1 | 5 | 1 | |
| Cajeput oil | 0.5 | 3 | 1 | 2 | 1 |
| Pentylene glycol | 5 | 8 | 8 | 5 | 8 |
| Panthenol | 5 | 3 | 1 | 3 | 4 |
| Sodium hyaluronate <50 | | 0.5 | | | |
| kilodaltons | | | | | |
| Sodium hyaluronate | | | | | |
| 1800 kilodaltons | | | | | |
| Sodium hyaluronate | | | 1 | | |
| 400 kilodaltons | | | | | |
| Hydrolysed sodium | | | | 2.5 | |
| hyaluronate | | | | | |
| Sodium hyaluronate | 2 | | | | 0.25 |
| crosspolymer | | | | | |
| Acetylglucosamine | | | | | 3 |
| Polyhydroxystearic acid | 1 | 1 | 1 | 1 | 1 |
| Iron oxide black | 35 | | | | |
| CI 77499 | | | | | |
| Iron oxide red CI 77491, | | 25 | | | |
| Iron oxide black | | | | | |
| CI 77499 | | | | | |
| Zinc oxide CI 77947, | | | 45 | | |
| boron nitride | | | | | |
| Iron oxide yellow CI | | | | 40 | |
| 77492 | | | | | |
| Ultramarine blue | | | | | 35 |
| CI 77007, | | | | | |
| kaolin | | | | | |

The ink is produced using the one-pot process, i.e., all raw materials are weighed together and homogenized at 25° C. using an UltraTurrax T25 from IKA for one minute at 5000 rpm. The tattoo ink is then ready and can be used.

The embodiments shown here are only examples of the present invention and should therefore not be understood to be limiting. Alternative embodiments contemplated by the skilled person are equally encompassed by the scope of protection of the present invention.

What is claimed is:

1. A tattoo ink based on a dispersion of at least one pigment in an aqueous carrier liquid,
wherein the tattoo ink contains a combination of at least one oil selected from a ginger oil and cajeput oil and hyaluron and/or one or more hyaluron derivatives.

2. The tattoo ink according to claim 1,
wherein the sum of the proportions of the at least one oil and of hyaluron and/or one or more hyaluron derivatives in the tattoo ink is from 0.1 to 11% by weight, in particular from 1 to 8% by weight and particularly preferably from 2.5 to 7% by weight.

3. The tattoo ink according to claim 1,
wherein the tattoo ink contains 0.05 to 10% by weight, preferably 0.05 to 7% by weight and in particular 0.5 to 5% by weight of ginger oil and/or 0.01 to 10% by weight, preferably 0.01 to 7% by weight and in particular 0.5 to 3% by weight of cajeput oil and 0.01 to 10% by weight, preferably 0.01 to 7% by weight and in particular 0.5 to 3% by weight of cajeput oil and 0.01 to 10% by weight, preferably 0.01 to 7% by weight and in particular 0.5 to 5% by weight hyaluron and/or one or more hyaluron derivatives.

4. The tattoo ink according to claim 1,
wherein the tattoo ink contains a ginger oil, cajeput oil and hyaluron and/or one or more hyaluron derivatives.

5. The tattoo ink according to claim 1,
wherein the hyaluron is hyaluronic acid or a physiologically compatible salt of hyaluronic acid, preferably sodium hyaluronate.

6. The tattoo ink according to claim 1,
wherein the one or more hyaluronic acid derivatives are selected from the group consisting of hydroxypropyl trimonium hyaluronate, hydrolysed sodium hyaluronate, vegan hyaluronic acid Tremella Fuciformis Sporocarp Extract, sodium acetylated hyaluronate and sodium hyaluronate crosspolymer.

7. The tattoo ink according to claim 1,
wherein the tattoo ink contains one or more hyaluronic acid derivatives and N-acetylglucosamine.

8. The tattoo ink according to claim 7,
wherein the tattoo ink contains 0.5 to 5% by weight of N-acetylglucosamine.

9. The tattoo ink according to claim 1,
wherein the ginger oil is plai oil.

10. The tattoo ink according to claim 1,
wherein the aqueous carrier liquid is water, a vegetable hydrolate or a mixture of water or a vegetable hydrolate and one or more physiologically harmless solvents, in particular lower saturated alcohols or particularly preferably glycerol.

11. The tattoo ink according to claim 1,
wherein the proportion of the at least one pigment in the tattoo ink is 1 to 90% by weight, particularly preferably 5 to 50% by weight and in particular 10 to 45% by weight.

12. The tattoo ink according to claim 1,
wherein the tattoo ink contains as pigment a white pigment from the group titanium dioxide, zinc oxides and boron nitride as carrier pigment in combination with at least one inorganic color pigment.

13. The tattoo ink according to claim 12,
wherein the tattoo ink contains boron nitride as carrier pigment.

14. The tattoo ink according to claim 1,
wherein the tattoo ink comprises a composition of:
40 to 70% by weight of aqueous carrier liquid;
2.5 to 8% by weight of the combination of ginger oil, cajeput oil and hyaluron and/or one or more hyaluron derivatives;
10 to 40% by weight of at least one pigment; and
0 to 15% by weight of auxiliary substances;
by weight.

15. Use of a tattoo ink according to claim 1 for producing a permanent tattoo on the skin of a living being.

16. Use of a tattoo ink according to claim 1 as a leave-on ink for temporarily coloring the skin of a living being or parts thereof.

17. A method for the production of tattoos,
wherein a suitable amount of a tattooing ink according to claim 1 is applied to the needles of a tattooing machine, in particular a magnetic coil machine or a rotary tattooing machine, and is introduced into the skin of a living being by means of the tattooing machine.
